SEQUENCE LISTING (7 sequences)

SEQ ID NO: 1 - DNA, 1320 bp, Homo sapiens
atgaagtcgatcctagatggccttgcagataccaccttccgcaccatcaccactgacctcctgggaagtcccttccaagagaagatgactgcgggagacaacccccagctagtcccagcagaccaggtgaacattacagaattttacaacaagtctctctcgtccttcaaggagaatgaggagaacatccagtgtggggagaacttcatggacatagagtgtttcatggtcctgaaccccagccagcagctggccattgcagtcctgtccctcacgctgggcaccttcacggtcctggagaacctcctggtgctgtgcgtcatcctccactcccgcagcctccgctgcaggccttcctaccacttcatcggcagcctggcggtggcagacctcctggggagtgtcatttttgtctacagcttcattgacttccacgtgttccaccgcaaagatagccgcaacgtgtttctgttcaaactgggtggggtcacggcctccttcactgcctccgtgggcagcctgttcctcacagccatcgacaggtacatatccattcacaggcccctggcctataagaggattgtcaccaggcccaaggccgtggtagcgttttgcctgatgtggaccatagccattgtgatcgccgtgctgcctctcctgggctggaactgcgagaaactgcaatctgtttgctcagacattttcccacacattgatgaaacctacctgatgttctggatcggggtcaccagcgtactgcttctgttcatcgtgtatgcgtacatgtatattctctggaaggctcacagccacgccgtccgcatgattcagcgtggcacccagaagagcatcatcatccacacgtctgaggatgggaaggtacaggtgacccggccagaccaagcccgcatggacattaggttagccaagaccctggtcctgatcctggtggtgttgatcatctgctggggccctctgcttgcaatcatggtgtatgatgtctttgggaagatgaacaagctcattaagacggtgtttgcattctgcagtatgctctgcctgctgaactccaccgtgaaccccatcatctatgctctgaggagtaaggacctgcgacacgctttccggagcatgtttccctcttgtgaaggcactgcgcagcctctggataacagcatgggggactcggactgcctgcacaaacacgcaaacaatgcagccagtgttcacagggccgcagaaagctgcatcaagagcacggtcaagattgccaaggtaaccatgtctgtgtccacagacacgtctgccgaggctctgtga

SEQ ID NO: 2 - protein, 439 aa, Homo sapiens
MKSILDGLADTTFRTITTDLLGSPFQEKMTAGDNPQLVPADQVNITEFYNKSLSSFKENEENIQCGENFMDIECFMVLNPSQQLAIAVLSLTLGTFTVLENLLVLCVILHSRSLRCRPSYHFIGSLAVADLLGSVIFVYSFIDFHVFHRKDSRNVFLFKLGGVTASFTASVGSLFLTAIDRYISIHRPLAYKRIVTRPKAVVAFCLMWTIAIVIAVLPLLGWNCEKLQSVCSDIFPHIDETYLMFWIGVTSVLLLFIVYAYMYILWKAHSHAVRMIQRGTQKSIIIHTSEDGKVQVTRPDQARMDIRLAKTLVLILVVLIICWGPLLAIMVYDVFGKMNKLIKTVFAFCSMLCLLNSTVNPIIYALRSKDLRHAFRSMFPSCEGTAQPLDNSMGDSDCLHKHANNAASVHRAAESCIKSTVKIAKVTMSVSTDTSAEAL

SEQ ID NO: 3 - DNA, 21 bp, Homo sapiens
tatgaagtcgatcctagatgg

SEQ ID NO: 4 - DNA, 19 bp, Homo sapiens
gttctccccacactggatg

SEQ ID NO: 5 - DNA, 20 bp, Homo sapiens
aattcttttcctgtgctgcc

SEQ ID NO: 6 - DNA, 48 bp, Artificial Sequence
  sequence of oligonucleotide used to delete polynucleotides encoding human CB1b receptor N-terminus
cgcaccatcaccactgacctcctgggaagtcccttccaagagaagatg

SEQ ID NO: 7 - DNA, 40 bp, Artificial Sequence
  sequence of oligonucleotide used to delete polynucleotides encoding human CB1b receptor N-terminus
gctccttcggtcctccgatatctgtcagaagtaagttggc

(19) United States
(12) Patent Application Publication
(10) Pub. No.: US 2006/0115816 A1
(43) Pub. Date: Jun. 1, 2006

(54) Splice variant cannabinoid receptor (cb1b)

(75) Inventors: Huy Khang Vu, Quebec (CA); Thierry Groblewski, Quebec (CA); Peter Greasly, Molndal (SE)

Correspondence Address: ASTRAZENECA R&D BOSTON, 35 GATEHOUSE DRIVE, WALTHAM, MA, 02451-1215, US

(73) Assignee: AstraZeneca A B, Sodertalje (SE)

(21) Appl. No.: 10/521,428

(22) PCT Filed: Jul. 14, 2003
(86) PCT No.: PCT/GB03/03067

§ 371 Date: Jan. 14, 2005

(30) Foreign Application Priority Data
Jul. 17, 2002 (SE) 0202240-8

(51) Int. Cl. C12Q 1/68; C07H 21/04; C12P 21/06; C07K 14/705

(52) U.S. Cl. 435/6; 435/69.1; 435/320.1; 435/325; 530/350; 536/23.5

(57) ABSTRACT

The invention is based on the identification of a novel splice variant of human cannabinoid 1 (CB1) receptor, termed CB1b, and its use in the identification of therapeutic agents and in the diagnosis, prevention, and treatment of CB associated disorders such as obesity, psychiatric, pain and neurological disorders.

### CROSS-REFERENCE TO RELATED APPLICATIONS

This application is a national stage filing under 35 U.S.C. 371 of International Application No. PCT/GB2003/003067, filed Jul. 14, 2003, which claims priority from Sweden Application No. 0202240-8, filed Jul. 17, 2002, the specification of which is incorporated by reference herein. International Application No. PCT/GB2003/003067 was published under PCT Article 21(2) in English.

### FIELD OF THE INVENTION

This invention relates to nucleic acid and amino acid sequences of a variant cannabinoid 1b (CB1b) receptor and to the use of these sequences in the diagnosis, prevention, and treatment of CB associated disorders such as obesity, psychiatric and neurological disorders.

### BACKGROUND OF THE INVENTION

Preparations of Cannabis sativa have been used for medicinal and recreational purposes for at least 4,000 years. Recently, cannabinoids have been the subject of renewed interest for their potential medicinal applications, e.g., in analgesia, nausea and appetite stimulation.

Cannabinoids exert their effects by binding to specific receptors located in the cell membrane. Two types of high-affinity cannabinoid receptors have been identified by molecular cloning: 1) CB1 receptors (Devane et al. Mol. Pharmacol. 1988, 34:605-613; Matsuda et al. Nature 1990, 346:561-564; Shire et al. J. Biol. Chem. 1995, 270:3726-3731; Ishac et al. Br. J. Pharmacol. 1996, 118:2023-2028), and 2) CB2 receptors (Munro et al. Nature 1993, 365:61-65). A splice variant of the CB1 receptor, CB1a has been identified. This variant is missing an amino terminal portion and contains a novel amino terminus (Shire D. et al., J. Biol. Chem., 270:3726-3731, 1995).

Both CB1 and CB2 are coupled to the same signal transduction pathway via the G-protein Gi. Activation of the canabinoid receptors leads to inhibition of adenylate cyclase and activation of MAP kinase. CB1 receptors can also modulate ion channels, inhibiting N—, and P/R-type calcium channels, stimulating inwardly rectifying K⁺ channels and enhancing the activation of the A-type K⁺ channel.

CB1 receptors are primarily but not exclusively expressed in the CNS and are believed to mediate the CNS effects of endogenous (e.g., anandamide) and exogenously applied cannabinoids. CB2 receptor expression is however restricted to the periphery (spleen>tonsils>immune cells).

### SUMMARY OF THE INVENTION

The invention is directed to a novel splice variant of the CB1 receptor, referred to herein as the CB1b receptor. Accordingly, the invention provides an isolated nucleic acid molecule having the nucleic acid sequence of the CB1b receptor of SEQ ID NO: 1, or variants or fragments thereof. The invention also provides a nucleic acid molecule comprising the complement of SEQ ID NO: 1, or variants or fragments thereof. In one embodiment, the present invention features an expression vector containing the claimed nucleic acid molecule. In yet another embodiment, the expression vector containing the claimed nucleic acid molecule is contained within a host cell.

In another aspect, the invention features a substantially purified polypeptide of the CB1b receptor having the amino acid sequence of SEQ ID NO: 2, or fragments or variants thereof.

In another aspect, the invention provides an isolated and substantially purified nucleic acid molecule encoding the polypeptide comprising the amino acid sequence of SEQ ID NO: 2, or fragments or variants thereof. The invention further provides a nucleic acid molecule comprising the complement of the nucleotide sequence encoding the amino acid sequence of SEQ ID NO: 2, or fragments of said nucleotide sequence.

In still another aspect, the invention provides a method for producing a polypeptide comprising the amino acid sequence of SEQ ID NO: 2, or fragments or variants thereof. The method includes: a) culturing the host cell containing an expression vector containing a nucleic acid sequence which encodes the CB1b receptor, or fragments or variants thereof, under conditions suitable for the expression of the receptor, or fragments or variants thereof; and b) recovering the receptor, or fragments or variants thereof, from the host cell culture.

The invention further provides a purified antagonist of the polypeptide of SEQ ID NO: 2, or fragments or variants thereof. In one aspect the invention provides a purified antibody which binds to a polypeptide comprising the amino acid sequence of SEQ ID NO: 2. In another aspect, the invention features an antisense nucleic acid targeted against the CB1b receptor mRNA described herein.

Still further, the invention provides a purified agonist of the polypeptide of SEQ ID NO: 2.

The invention further provides a pharmaceutical composition comprising an isolated inhibitory nucleic acid molecule capable of selectively binding to nucleic acid possessing the sequence disclosed in SEQ ID NO: 1, in conjunction with a suitable pharmaceutical carrier. The invention further provides a pharmaceutical composition comprising an agonist or an antagonist of the CB1b receptor.

The invention also provides a method for treating or preventing a CB associated disorder, e.g., for the treatment of obesity, pain, psychiatric disorders such as psychotic disorders, anxiety, anxio-depressive disorders, depression, cognitive neurological disorders such as dementia, multiple sclerosis, Raynaud's syndrome, Parkinson's disease, Huntington's chorea and Alzheimer's disease, comprising administering to a subject in need of such treatment an effective amount of a pharmaceutical composition described herein.

The invention also provides a method for detecting a polynucleotide which encodes a CB1b receptor in a biological sample comprising the steps of: a) hybridizing the complement of the polynucleotide sequence which encodes SEQ ID NO: 2 to a nucleic acid material of a biological sample, thereby forming a hybridization complex; and b) detecting the hybridization complex, wherein the presence of the complex correlates with the presence of a polynucleotide encoding a CB1b receptor in the biological sample.

The term “agonist”, as used herein, refers to a molecule which, when bound to CB1b receptor, increases or prolongs the duration of the effect of CB1b receptor. Agonists may include proteins, nucleic acids, carbohydrates, or any other molecules which bind to and modulate the effect of the CB1b receptor.

The term “antagonist”, as used herein, refers to a molecule which, when bound to the CB1b receptor, decreases the amount or the duration of the effect of the biological or immunological activity of the CB1b receptor. Antagonists may include proteins, nucleic acids, carbohydrates, antibodies or any other molecules which decrease the effect of CB1b receptor.

The term “substantially purified”, as used herein, refers to nucleic or amino acid sequences that are removed from their natural environment, isolated or separated, and are at least 60% free, preferably 75% free, and most preferably 90% free from other components with which they are naturally associated. Techniques for purifying polynucleotides of interest are well-known in the art and include, for example, disruption of the cell containing the polynucleotide with a chaotropic agent and separation of the polynucleotide(s) and proteins by ion-exchange chromatography, affinity chromatography and sedimentation according to density. Methods for purifying proteins are known in the art.

The term “isolated” means that the material is removed from its original environment (e.g., the natural environment if it is naturally occurring). For example, a naturally-occurring polynucleotide or polypeptide present in a living animal is not isolated, but the same polynucleotide or DNA or polypeptide, which is separated from some or all of the coexisting materials in the natural system, is isolated. Such polynucleotide could be part of a vector and/or such polynucleotide or polypeptide could be part of a composition, and still be isolated in that the vector or composition is not part of its natural environment.

Unless defined otherwise, all technical and scientific terms used herein have the same meanings as commonly understood by one of ordinary skill in the art to which this invention belongs. All publications mentioned herein are incorporated herein by reference. It is understood that this invention is not limited to the particular methodology, protocols, cell lines, vectors, and reagents described, as these may vary.

### DESCRIPTION OF THE INVENTION

The invention is based on the discovery of a splice variant of the human CB1 receptor. The variant is referred to herein as CB1b. CB1b and has a deletion of exactly 99 nucleotides at the N-terminal from positions 64 to 162 compared to the sequence of wildtype human CB1 receptor.

The CB1b splice variant has been found to have an altered functional activity in response to various established CB1 agonists. To date the function of cannabinoid ligands has been assumed to be at either the CB1 or CB2 receptors. The identification of the CB1b splice variant suggests that some or all of the activities of ligands active at CB receptors may be, at least in part, due to their activity at CB1b receptors. In order to develop a specific CB receptor agonist or antagonist, or gene therapy agent, it is essential that an understanding of the actual contribution of a particular receptor is known. For example, in the development of a full antagonist of CB1 it is critical to know whether that antagonist also acts as an antagonist against CB1b.

CB1b Nucleic Acid Sequence and Polypeptide

The invention encompasses a CB1b receptor having at least 85%, e.g., 90%, 95%, 96%, 97%, 98% or 99%, sequence identity to the CB1b receptor sequence of SEQ ID NO: 1. The comparison of sequences and determination of percent sequence identity between two sequences can be accomplished using a mathematical algorithm. In a preferred embodiment, the percent identity between two amino acid sequences is determined using the Needleman and Wunsch (J. Mol. Biol. (48):444-453 (1970)) algorithm which has been incorporated into the GAP program in the GCG software package (available at http://www.gcg.com), using either a Blosum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at ttp://www.gcg.com), using a NWSgapdna.CMP matrix and a gap weight of 40, 50, 60, 70, or 80 and a length weight of 1, 2, 3, 4, 5, or 6. For the purpose of this invention the percent identity between two amino acid or nucleotide sequences is determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4.

The invention also encompasses polynucleotides which encode the CB1b receptor of is SEQ ID NO: 2, and variants thereof. Accordingly, any nucleic acid sequence which encodes the amino acid sequence of the splice variant can be used to produce recombinant molecules which express the CB1b receptor. It will be appreciated by those skilled in the art that as a result of the degeneracy of the genetic code, a multitude of nucleotide sequences encoding CB1b receptor, some bearing minimal homology to the nucleotide sequences of any known and naturally occurring gene, may be produced. Thus, the invention contemplates each and every possible variation of nucleotide sequence that could be made by selecting combinations based on possible codon choices. These combinations are made in accordance with the standard triplet genetic code as applied to the nucleotide sequence of naturally occurring CB1b receptor, and all such variations are to be considered as being specifically disclosed.

The invention also encompasses production of DNA sequences, or fragments thereof, which encode the CB1b receptor and its derivatives, entirely by synthetic chemistry. The polypeptides of the invention can be synthesised chemically. For example, by the Merryfield technique (J. Amer. Chem. Soc. 85:2149-2154, 1968). Numerous automated polypeptide synthesisers, such as Applied Biosystems 431A Peptide Synthesizer also now exist. After production, the synthetic sequence may be inserted into any of the many available expression vectors and cell systems using reagents that are well known in the art.

Also encompassed by the invention are polynucleotide sequences that are capable of hybridizing to the claimed CB1b receptor, and in particular, those shown in SEQ ID NO: 1, under various conditions of stringency as taught in Wahl, G. M. and S. L. Berger (1987; Methods Enzymol. 152:399407) and Kimmel, A. R. (1987; Methods Enzymol. 152:507-511). Appropriate stringency conditions which promote DNA hybridization, for example, 6.0×sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6.3.6. For example, the salt concentration in the wash step can be selected from a low stringency of about 2.0 SSC at 50° C. to a high stringency of about 0.2×SSC at 50° C. In addition, the temperature in the wash step can be increased from low stringency conditions at room temperature, about 22° C., to high stringency conditions at about 65° C. Moderately stringent conditions are, for example at about 2.0×SSC and about 40° C.

Also included in the invention are CB1b receptor polypeptides having at least 95% amino acid sequence identity to the CB1b receptor of SEQ ID NO: 2 and which variants retain the potencies and efficacies of the compounds listed in Table 1. A most preferred is CB1b receptor variant is one having at least 96% 97%, 98% or 99% amino acid sequence identity to SEQ ID NO: 2.

According to a further aspect of the invention there is provided an isolated polypeptide having at least 95% sequence identity to SEQ ID NO:2, which polypeptide lacks the 33 amino acid stretch from amino acids 22-54 inclusive of wild-type CB1 receptor.

According to a further aspect of the invention there is provided an isolated nucleic acid comprising a nucleotide sequence which encodes a CB1 receptor variant having at least 95% sequence identity to SEQ ID NO:1, which nucleotide sequence lacks the 99 bases from position 64-162 inclusive of wild-type CB1 receptor.

The invention also includes variants of the CB1b receptor which can contain one or more substitutions of amino acid residues which result in a silent change and a functionally equivalent CB1b receptor. Deliberate amino acid substitutions may be made on the basis of similarity in polarity, charge, solubility, hydrophobicity, hydrophilicity, and/or the amphipathic nature of the residues as long as the biological or immunological activity of CB1b receptor is retained. For example, negatively charged amino acids may include aspartic acid and glutamic acid; positively charged amino acids may include lysine and arginine; and amino acids with uncharged polar head groups having similar hydrophilicity values may include leucine, isoleucine, and valine, glycine and alanine, asparagine and glutamine, serine and threonine, and phenylalanine and tyrosine.

In order to express a biologically active CB1b receptor, the nucleotide sequences encoding a CB1b receptor or functional equivalents, may be inserted into an appropriate expression vector, i.e., a vector which contains the necessary elements for the transcription and translation of the inserted coding sequence. Methods which are well known to those skilled in the art may be used to construct expression vectors containing sequences encoding the CB1b receptor and appropriate transcriptional and translational control elements. These methods include in vitro recombinant DNA techniques, synthetic techniques, and in vivo genetic recombination. Such techniques are described in Sambrook, J. et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York, N.Y.

A variety of expression vector/host systems may be utilized to contain and express sequences encoding the CB1b receptor. These include, but are not limited to, microorganisms such as bacteria transformed with recombinant bacteriophage, plasmid, or cosmid DNA is expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV); bacterial expression vectors (e.g., Ti or pBR322 plasmids); or with animal cell systems. The invention is not limited by the host cell employed. When producing the polypeptide by recombinant expression in heterologous host strains, it may be desirable to adopt the codon usage (preference) of the host organism (Murray. N.A.R. 17:477-508, 1989).

The “control elements” or “regulatory sequences” are those non-translated regions of the vector (enhancers, promoters, 5′ and 3′ untranslated regions) which interact with host cellular proteins to carry out transcription and translation. Such elements may vary in their strength and specificity.

Host cells transformed with nucleotide sequences encoding the CB1b receptor may be cultured under conditions suitable for the expression and recovery of the protein from the cell culture. The protein produced by a transformed cell may be secreted or contained intracellularly depending on the sequence and/or the vector used.

In another embodiment of the invention, natural, modified, or recombinant nucleic acid sequences encoding the CB1b receptor may be ligated to a heterologous sequence to encode a fusion protein. For example, to screen peptide libraries for inhibitors of CB1b receptor activity, it may be useful to encode a chimeric CB1b receptor protein that can be recognized by a commercially available antibody. A fusion protein may also be engineered to contain a cleavage site located between the CB1b receptor encoding sequence and the heterologous protein sequence, so that CB1b receptor may be cleaved and purified away from the heterologous moiety.

In another embodiment, the protein itself may be produced using chemical methods to synthesize the amino acid sequence of the CB1b receptor, or a fragment thereof. For example, peptide synthesis can be performed using various solid-phase techniques (Roberge, J. Y. et al. (1995) Science 269:202-204) and automated synthesis may be achieved, for example, using the ABI 431A Peptide Synthesizer (Perkin Elmer). The newly synthesized peptide may be substantially purified by preparative high performance liquid chromatography (e.g., Creighton, T. (1983) Proteins, Structures and Molecular Principles, W H Freeman and Co., New York, N.Y.).

Drug Screening

In one screening method, a cell-based assay in which a cell, or cell-membrane, which is expresses a CB1b receptor, variant or biologically active portion thereof, is contacted with a test compound in the presence or absence of a CB1b receptor ligand (such as any of the active compounds in Table 1) and the ability of the test compound to modulate CB1b receptor activity in the presence of the CB1b receptor ligand is determined. Determining the ability of the test compound to modulate the ability of the CB1b receptor to bind to a CB1b receptor ligand such as CB can be accomplished, for example, by coupling the CB with a radioisotope or enzymatic label such that binding of the CB to the CB1b receptor can be determined by detecting the labeled CB in a complex. For example, a CB1b receptor ligand can be labelled with ¹²⁵I, ³⁵S, ¹⁴C, or ³H, either directly or indirectly, and the radioisotope detected by direct counting of radioemmission or by scintillation counting. Alternatively, fluorescently labelled ligands could be used.

In another embodiment, an assay is a cell-based assay comprising contacting a cell expressing a CB1b receptor. The method can be used to identify a modulator of CB1b activity. For example, the method can include adding a test compound, and determining the ability of the test compound to inhibit or stimulate the activity of the CB1b receptor. In one example where the test compound is tested for its ability to act as an inhibitor, the method includes stimulating the receptor with an agonist (e.g. Δ9-THC), then adding a test compound, and finally determining the ability of the test compound to inhibit the activity of the CB1b receptor. Determining the ability of the test compound to inhibit or stimulate a CB1b receptor can be accomplished by detecting induction of a cellular second messenger. Similar to other CB receptors, CB1b is likely to be coupled to the transduction pathway via the G-protein Gi. Thus, the ability of a test compound to modulate the activity of a second messenger such as adenylate cyclase, MAP kinase, or Ca²⁺ can be used to determine if the test compound is an inhibitory or stimulatory agent.

Alternatively, the method can include detecting the induction of a reporter gene which includes a CB1b receptor target-responsive regulatory element operatively linked to a nucleic acid encoding a detectable marker, e.g., luciferase.

In another embodiment, inhibitory or stimulatory agents of CB1b receptor expression are identified in a method wherein a cell is contacted with a test compound and the expression of CB1b receptor mRNA or protein in the cell is determined. The level of expression of CB1b receptor mRNA or protein in the presence of the test compound is compared to the level of expression of CB1b receptor mRNA or protein in the absence of the test compound. The test compound can then be identified as an inhibitor or stimulator of CB1b receptor expression based on this comparison. In one example, inhibitory agents of CB1b agonist-stimulated receptor expression are identified in a method wherein a cell is contacted with a test compound and an agonist and the expression of CB1b receptor mRNA or protein in the cell is determined and compared to the level of expression of the CB1b receptor mRNA or protein in the presence of just the agonist. A decrease in mRNA or protein levels in the presence of the test compound compared to the levels in the absence of the inhibitory agent indicates that the agent is an inhibitor.

In another method, the method is a non-cell based method. In this assay, a CB1b receptor is contacted with a test compound in the presence of a CB receptor ligand and the ability of the test inhibitory agent to inhibit the binding of the CB1b receptor to the CB1b receptor ligand is determined.

High throughput screening of compounds can also be used to identify a compound which binds the CB1b receptor, as described in published PCT application WO84/03564. In this method, as applied to a CB1b receptor, large numbers of different small test compounds are synthesized on a solid substrate, such as plastic pins or some other surface. The test compounds are reacted with CB1b receptor, variant or fragments thereof, and washed. Bound CB1b receptor is then detected by methods well known in the art. Purified CB1b receptor can also be coated directly onto plates for use in the aforementioned drug screening techniques. Alternatively, non-neutralizing antibodies can be used to capture the peptide and immobilize it on a solid support.

In another embodiment, one may use competitive drug screening assays in which neutralizing antibodies capable of binding CB1b receptor specifically compete with a test compound for binding to the CB1b receptor. In this manner, the antibodies can be used to detect the presence of any peptide which shares one or more antigenic determinants with the CB1b receptor.

According to another aspect of the invention there is provided a screening system wherein the modulatory ability of a test compound is determined by screening the compound against a panel of cannabinoid receptors, said panel comprising CB1b and at least one other cannabinoid receptor family member. Any of the screening methods disclosed herein could be used in this aspect of the invention.

In a particular embodiment, the “at least one other cannabinoid receptor family member” is selected from the group consisting of CB1, CB1a and CB2.

In a particular embodiment the modulatory effects of the test compound is measured against CB1b and at least CB1 and CB1a.

In a particular embodiment the modulatory effects of the test compound is measured against CB1b and at least CB1, CB2 and CB1a.

According to a further aspect of the invention there is provided a method for determining the selectivity of a test compound against a cannabinoid receptor family member comprising determining the ability of the test compound to modulate each of a panel of cannabinoid receptors, said cannabinoid receptor panel comprising the CB1b receptor and at least one other cannabinoid receptor selected form CB1, CB2 and CB1a.

A profile of the effects of the test compound against each receptor can then be generated.

Given the diverse roles of cannabinoids in biological processes, the asays of the present invention are useful for the identification and development of compounds with selective effect profiles.

Pathway mapping may also be used to determine each protein in the cell with which the CB1b receptor interacts and, in turn, the proteins with which each of these proteins interacts also. In this way it is possible to identify the specific critical signaling pathway which links the disease stimulus to the cell's response thereby enabling the identification of new potential targets for therapy intervention. According to a further aspect of the invention there is provided the use of the CB1b receptor or a fragment thereof in research to identify further gene targets implicated in CB associated disorders.

Therapeutics

The CB1 receptor has been associated with a number of conditions and is believed to play a role in altered pain perception, cognition and memory, addiction/substance abuse, schizophrenia/psychosis/delusion disorders, psychological, neurological, neurodegenerative, stress, mood modulation, depressive, anxiety, blood pressure regulation, gastrointestinal, eating and appetite disorders. Indeed, drugs known to interact with CB1 receptor have been claimed useful in the treatment or prevention of many of the aforementioned conditions. It is also envisaged that drugs which interact with CB1b will also be useful for the treatment of CB associated disorders.

In one embodiment, the CB1b receptor, agonist, inverse agonist, modulator or antagonist may be administered to a subject to treat a CB associated disorder including obesity, psychiatric disorders such as psychotic disorders, anxiety, anxio-depressive disorders, depression, cognitive neurological disorders such as dementia, multiple sclerosis, Raynaud's syndrome, Parkinson's disease, Huntington's chorea and Alzheimer's disease. A true antagonist or inverse agonist of the CB1b receptor are also potentially useful for the treatment of immune cardiovascular, reproductive and endocrine disorders, and also diseases related to the respiratory and gastronintestinal systems.

It may be of clinical interest to detect the level of CB1b in a test sample. A variety of protocols for detecting and measuring the expression of the CB1b receptor, using for example, polyclonal or monoclonal antibodies specific for the protein are known in the art. Examples include enzyme-linked immunosorbent assay (ELISA), radioimmunoassay (RIA), and fluorescence activated cell sorting (FACS). A two-site, monoclonal-based immunoassay utilizing monoclonal antibodies reactive to two non-interfering epitopes on CB1 is preferred, but a competitive binding assay may be employed. These and other assays are described, among other places, in Hampton, R. et al. (1990; Serological Methods, a Laboratory Manual, APS Press, St Paul, Minn.) and Maddox, D. E. et al. (1983; J. Exp. Med. 158:1211-1216).

Antibodies to CB1b receptor may be generated using methods that are well known in the art. Such antibodies may include, but are not limited to, polyclonal, monoclonal, chimeric, single chain, Fab fragments, and fragments produced by a Fab expression library. Neutralizing antibodies, (i.e., those which inhibit dimer formation) are especially preferred for therapeutic use. Monoclonal antibodies to specific antigens may be obtained by methods known to those skilled in the art, such as from hybridoma cells, phage display libraries or other methods. Monoclonal antibodies may be inter alia, human, rat or mouse derived. For the production of human monoclonal antibodies, hybridoma cells may be prepared by fusing spleen cells from an immunised animal, e.g. a mouse, with a tumour cell. Appropriately secreting hybridoma cells may thereafter be selected (Koehler & Milstein, Nature 256:495-497 (1975); Cole et al., “Monoclonal antibodies and Cancer Therapy”, Alan R Liss Inc, New York N.Y. pp 77-96 (1985)). Such antibodies may be of any immunoglobulin class including IgG, IgM, IgE, IgA, IgD and any subclass thereof. Polyclonal antibodies can be generated by immunisation of an animal (such as a mouse, rat, goat, horse, sheep etc) with a CB1 splice variant receptor as the antigen.

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between CB1b receptor and its specific antibody. A two-site, monoclonal-based immunoassay utilizing monoclonal antibodies reactive to two non-interfering CB1b receptor epitopes is preferred, but a competitive binding assay may also be employed (Maddox, supra).

In another embodiment of the invention, the polynucleotides encoding CB1b receptor, variants or any fragment thereof, may be used for therapeutic purposes as “inhibitory nucleic acid molecules”. In one aspect, the complement of the polynucleotide encoding CB1b receptor may be used in situations in which it would be desirable to block the transcription of the mRNA. In particular, cells may be transformed with sequences complementary to polynucleotides encoding CB1b receptor. Thus, complementary molecules or fragments may be used to modulate CB1b receptor activity, or to achieve regulation of gene function. Such technology using sense or antisense oligonucleotides or larger fragments, can be designed from various locations along the coding or control regions of sequences encoding CB1b receptor. In each of the above aspects of the invention, the “inhibitory nucleic acid molecule” is selected from the group consisting of: an antisense, ribozyme, triple helix aptemer and RNAi molecule.

Expression vectors derived from retro viruses, adenovirus, herpes or vaccinia viruses, or from various bacterial plasmids may be used for delivery of nucleotide sequences to the targeted organ, tissue or cell population. Methods which are well known to those skilled in the art can be used to construct vectors which will express nucleic acid sequence which is complementary to the polynucleotides of the gene encoding CB1b receptor. These techniques are described both in Sambrook et al. (supra) and in Ausubel et al. (supra).

Ribozymes, enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. Examples which may be used include engineered hammerhead motif ribozyme molecules that can specifically and efficiently catalyze endonucleolytic cleavage of sequences encoding CB1b receptor. The design, construction and use of such ribozymes is well known in the art and is more fully described in Haselhoff and Gerlach, (Nature. 334:585-591, 1988).

Specific ribozyme cleavage sites within any potential RNA target are initially identified by scanning the target molecule for ribozyme cleavage sites which include the following sequences: GUA, GUU, and GUC. Once identified, short RNA sequences of between 15 and 20 ribonucleotides corresponding to the region of the target gene containing the cleavage site may be evaluated for secondary structural features which may render the oligonucleotide inoperable. The suitability of candidate targets may also be evaluated by testing accessibility to hybridization with complementary oligonucleotides using ribonuclease protection assays.

Complementary ribonucleic acid molecules and ribozymes of the invention may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA sequences encoding CB1b receptor. Such DNA sequences may be incorporated into a wide variety of vectors with suitable RNA polymerase promoters such as T7 or SP6. Alternatively, these cDNA constructs that synthesize complementary RNA constitutively or inducibly can be introduced into cell lines, cells, or tissues.

RNA molecules may be modified to increase intracellular stability and half-life. Possible modifications include, but are not limited to, the addition of flanking sequences at the 5′ and/or 3′ ends of the molecule or the use of phosphorothioate or 2′ O-methyl rather than phosphodiesterase linkages within the backbone of the molecule. This concept is inherent in the production of PNAs and can be extended in all of these molecules by the inclusion of nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

In another aspect of the invention, oligonucleotides designed to hybridise to the 5′-region of the CB1b receptor gene so as to form triple helix structures may be used to block or reduce transcription of the CB1b receptor gene. In another alternative, RNA interference (RNAi) oligonucleotides or short (18-25 bp) RNAi CB1b receptor sequences cloned into plasmid vectors are designed to introduce double stranded RNA into mammalian cells to inhibit and/or result in the degradation of CB1b receptor messenger RNA. CB1b receptor RNAi molecules may begin adenine/adenine (AA) or at least (any base-A,U,C or G)A . . . and may comprise of 18 or 19 or 20 or 21 or 22 or 23, or 24 or 25 base pair double stranded RNA molecules with the preferred length being 21 base pairs and be specific to individual CB1b receptor sequences with 2 nucleotide 3′ overhangs or hairpin forming 45-50mer RNA molecules. The design, construction and use of such small inhibitory RNA molecules is well known in the art and is more fully described in the following: Elbashir et al., (Nature. 411(6836):494498, 2001); Elbashir et al., (Genes & Dev. 15:188-200, 2001); Harborth, J. et al. (J. Cell Science 114:4557-4565, 2001); Masters et al. (Proc. Natl. Acad. Sci. USA 98:8012-8017, 2001); and, Tuschl et al., (Genes & Dev. 13:3191-3197, 1999).

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection, by liposome injections or polycationic amino polymers (Goldman, C. K. et al. (1997) Nature Biotechnology 15:462-66; incorporated herein by reference) may be achieved using methods which are well known in the art.

Any of the therapeutic methods described above may be applied to any subject in need of such therapy, including, for example, mammals such as dogs, cats, cows, horses, rabbits, monkeys, and most preferably, humans.

Pharmaceutical Composition and Administration

The invention also relates to the administration of a pharmaceutical composition, in conjunction with a pharmaceutically acceptable carrier, for any of the therapeutic effects discussed above. Such pharmaceutical compositions may consist of CB1b receptor, antibodies to CB1b receptor, mimetics, agonists, antagonists, or inhibitors of CB1b receptor. The compositions may be administered alone or in combination with at least one other agent, such as stabilizing compound, which may be administered in any sterile, biocompatible pharmaceutical carrier, including, but not limited to, saline, buffered saline, dextrose, and water. The compositions may be administered to a patient alone, or in combination with other agents, drugs or hormones.

The pharmaceutical compositions utilized in this invention may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, or rectal means.

In addition to the active ingredients, these pharmaceutical compositions may contain suitable pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing Co., Easton, Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropylmethyl-cellulose, or sodium carboxymethylcellulose; gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores may be used in conjunction with suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations which can be used orally include push-fit capsules made of gelatin, as well as soft, sealed capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Pharmaceutical formulations suitable for parenteral administration may be formulated in aqueous solutions, preferably in physiologically compatible buffers such as Hanks' solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Non-lipid polycationic amino polymers may also be used for delivery. Optionally, the suspension may also contain suitable stabilizers or agents which increase the solubility of the compounds to allow for the preparation of highly concentrated solutions.

The pharmaceutical compositions of the present invention may be manufactured in a manner that is known in the art, e.g., by means of conventional mixing, dissolving, granulating, dragee-making, levigating, emulsifying, encapsulating, entrapping, or lyophilizing processes.

A therapeutically effective dose refers to that amount of active ingredient, for example CB1b receptor or fragments thereof, antibodies of CB1b receptor, agonists, antagonists or inhibitors of CB1b receptor, which ameliorates the symptoms or condition. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50. Pharmaceutical compositions which exhibit large therapeutic indices are preferred. The data obtained from cell culture assays and animal studies is used in formulating a range of dosage for human use. The dosage contained in such compositions is preferably within a range of circulating concentrations that include the ED50 with little or no toxicity. The dosage varies within this range depending upon the dosage form employed, sensitivity of the patient, and the route of administration.

The exact dosage will be determined by the practitioner, in light of factors related to the subject that requires treatment. Dosage and administration are adjusted to provide sufficient levels of the active moiety or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

Normal dosage amounts may vary from 0.1 to 100,000 micrograms, up to a total dose of about 1 g, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

Diagnostics

In another embodiment, antibodies which specifically bind CB1b receptor may be used for the diagnosis of conditions or diseases characterized by expression of CB1b receptor, or in assays to monitor patients being treated with CB1b receptor, agonists, antagonists or inhibitors. The antibodies useful for diagnostic purposes may be prepared in the same manner as those described above for therapeutics. Diagnostic assays for CB1b receptor include methods which utilize the antibody and a label to detect CB1b receptor in human body fluids or extracts of cells or tissues. The antibodies may be used with or without modification, and may be labeled by joining them, either covalently or non-covalently, with a reporter molecule. A wide variety of reporter molecules which are known in the art may be used, several of which are described above.

A variety of protocols including ELISA, RIA, and FACS for measuring CB1b receptor are known in the art and provide a basis for diagnosing altered or abnormal levels of CB1b receptor expression. Normal or standard values for CB1b receptor expression are established by combining body fluids or cell extracts taken from normal mammalian subjects, preferably human, with antibody to CB1b receptor under conditions suitable for complex formation.

In another embodiment of the invention, the polynucleotides encoding CB1b receptor may be used for diagnostic purposes. The polynucleotides which may be used include oligonucleotide sequences, complementary RNA and DNA molecules, and PNAs. The polynucleotides may be used to detect and quantitate gene expression in biopsied tissues in which expression of CB1b receptor may be correlated with disease. The diagnostic assay may be used to distinguish between absence, presence, and excess expression of CB1b receptor, and to monitor regulation of CB1b receptor levels during therapeutic intervention.

In one aspect, hybridization with PCR probes which are capable of detecting polynucleotide sequences, including genomic sequences, encoding CB1b receptor or closely related molecules, may be used to identify nucleic acid sequences which encode CB1b receptor. The specificity of the probe, whether it is made from a highly specific region, e.g., 10 unique nucleotides in the 5′ regulatory region, or a less specific region, e.g., especially in the 3′ coding region, and the stringency of the hybridization or amplification (maximal, high, intermediate, or low) will determine whether the probe identifies only naturally occurring is sequences encoding CB1b receptor, alleles, or related sequences.

Probes may also be used for the detection of related sequences, and should preferably contain at least 50% of the nucleotides from any of the CB1b receptor encoding sequences. The hybridization probes of the subject invention may be DNA or RNA and derived from the nucleotide sequence of SEQ ID NO: 1 or from genomic sequence including promoter, enhancer elements, and introns of the naturally occurring CB1b receptor.

Means for producing specific hybridization probes for DNAs encoding CB1b receptor include the cloning of nucleic acid sequences encoding CB1b receptor or CB1b receptor derivatives into vectors for the production of mRNA probes. Such vectors are known in the art, commercially available, and may be used to synthesize RNA probes in vitro by means of the addition of the appropriate RNA polymerases and the appropriate labeled nucleotides. Hybridization probes may be labeled by a variety of reporter groups, for example, radionuclides such as 32P or 35S, or enzymatic labels, such as alkaline phosphatase coupled to the probe via avidin/biotin coupling systems, and the like.

Polynucleotide sequences encoding CB1b receptor may be used for the diagnosis of conditions or disorders which are associated with expression of CB1b receptor. Examples of such conditions or disorders include obesity, psychiatric disorders such as psychotic disorders, anxiety, anxio-depressive disorders, depression, cognitive neurological disorders such as dementia, multiple sclerosis, Raynaud's syndrome, Parkinson's disease, Huntington's chorea and Alzheimer's disease, immune disorders, cardiovascular disorders, reproductive disorders, endocrine disorders, and respiratory disorders.

### EXAMPLES

The following examples are non-limiting and are given by way of illustration only. It will be appreciated by those skilled in the art that the examples are to be looked upon as guidelines, and the invention is not restricted to the exemplified compositions. A wide range of combinations is possible to give film coatings having the necessary properties required for each specific application.

### Example 1

### A New Variant of the Human CB1b Receptor

Using a PCR protocol a new amino terminal splice variant of the human CB1 receptor was identified.

The method included PCR detection of the amino terminal region as well as the whole coding region of human CB1 receptor. The oligonucleotides used were:

| | | |
|---|---|---|
| 5′-tatgaagtcgatcctagatgg-3′ | hCB1-F | (SEQ ID NO: 3) |
| 5′-gttctccccacactggatg-3′ | hCB1-3R | (SEQ ID NO: 4) |
| 5′-aattcttttcctgtgctgcc-3′ | hCB1-2R | (SEQ ID NO: 5) |

The final PCR product was obtained after two rounds of PCR amplification. In the first PCR round: the first two oligonucleotides were used in a polymerase chain reaction mixture of 50 μl, containing 2 μl of a pre-made human foetal brain cDNA library in pcDNA3.1+(Invitrogen, cat A550-24), 1×PCR buffer (10 mM tris-HCl, pH 9.0, 1.5 mM MgCl2 and 50 mM KCl), 0.4 mM dNTPs (Amersham-Pharmacia Biotech), 5 U Taq polymerase (Amersham-Pharmacia Biotech) and 50 pmoles of each hCB1-F and hCB1-3R primers. The amplification was carried out in a T-Gradient Thermo Block (Biometra). The template was denatured at 94° C. for 4 minutes, followed by 40 cycles consisting of denaturation (at 94° C. for 30 seconds), annealing (at 54° C. for 30 seconds) and extension (at 72° C. for 30 seconds) steps, then extended for an additional 4 minutes at 72° C. The resulting product was resolved on a 1% agarose gel and only a major band at 304 bp corresponding to the human un-spliced CB1 receptor was detected. The 0.1 Kb-0.25 Kb gel area was excised and purified with QIAquick gel extraction kit (Qiagen) for further PCR amplification.

A second PCR round using exactly the same conditions as described above was performed on the purified material and the resulting product was again resolved on a 1% agarose gel. A major band at 205 bp was isolated before subcloning into pGEMT Easy Vector (Promega). The plasmids were prepared with the alkaline lysis protocol using Qiaprep 8 kit (Qiagen) and screened by sequencing using ABI Prism dRhodamine cycle sequencing ready reaction kit (Perkin Elmer Applied Biosystems).

The sequencing showed a deletion of exactly 99 nucleotides at the N-terminal, comprising from positions 64 to 162 when comparing to the sequence of un-spliced human CB1 receptor. This deletion does not change the reading frame of the receptor.

### Example 2

### Generating a CB1b Receptor

The CB1b receptor coding sequence was created with the Transformer Site-Directed-Mutagenesis kit (Clontech, cat K1600-1) by deleting the 99 nucleotides (from nucleotides at position 64 to 162) in the N-terminal region of the CB1 receptor gene. As described in the kit instructions, the following procedure was followed: The template used was the human CB1 receptor gene cloned into pcDNA3 vector. The following oligonucleotides were used:

| | |
|---|---|
| 5′-cgcaccatcaccactgacctcctgggaagtcccttccaagagaagatg-3′ | (SEQ ID NO: 6) |
| hCB 12hCB1b receptor | |
| (deletion primer) | |
| 5′-gctccttcggtcctccgatatctgtcagaagtaagttggc-3′ | (SEQ ID NO: 7) |
| Pvu2EcoR5 | |
| (selection primer) | |

The selection restriction enzyme used was PvuI.

### Example 3

### Functional Assay

In order to assess the functionality of the human CB1b receptor variant, a functional assay was performed with FLIPR (Fluorescent Imaging Plate Reader, Molecular Devices) using the fluorescent calcium indicator Fluo-3 (Molecular Probes) on a 96 well platform. HEK-293S cells, stable pool expressing the un-spliced hCB1 receptor or the human CB1a receptor or the human CB1b receptor or the human CB2 receptor (all co-expressed with the promiscuous Gα16 subunit) were plated at a density of 24 000 cells/well in a 96 well plate. On the day of the experiment, the cells were initially washed once with Hank's buffer (pH7.4) containing 20 mM Hepes and 0.1% BSA (HBSS buffer) and subsequently loaded with fluorescent solution (HBSS buffer, 4 μM Fluor-3 and 0.04% pluronic acid). The cells were incubated at 37° C. for 1 hour in a humidified chamber. Following the incubation step, cells were washed five times with HBSS buffer. The cells were analyzed using the FLIPR system to measure the mobilization of intracellular calcium in response to the non-selective cannabinoid agonist HU-210. The results showed that the human CB1b receptor variant receptor is functional and responds to HU210 with an EC50=of 0.15 nM. Interestingly EC50 value obtained for HU-210 on the human CB1b receptor variant was lower than the one measured on the “classical” un-spliced human CB 1 receptor (EC50=0.06 nM). A range of other natural and synthetic cannabinoid ligands were also tested. The table below summarizes the agonist properties of some natural and synthetic cannabinoid ligands on CB1, CB1a, CB1b and CB2.

TABLE 1

| Agonist | hCB1 | hCB1a | hCB1b | hCB2 |
|---|---|---|---|---|
| JWH133 | >1000 | >1000 | >1000 | 1.8 |
| CP55940 | 0.28 | 1.2 | 0.46 | 0.2 |
| Anandamide | 32 | >1000 | >1000 | 99 |
| Noladin Ether | 5.1 | >1000 | >1000 | >1000 |
| WIN55-212 | 23 | 2.9 | 0.7 | 0.8 |
| Hu210 | 0.06 | 0.2 | 0.15 | 0.2 |
| JWH-015 | 374 | >1000 | >1000 | 4.2 |
| Δ9-THC | 3.9 | 2.9 | 3.6 | >1000 |
| Virodhamine | 934 | >1000 | >1000 | 1310 |
| 2-arachodonylglycerol | 228 | 744(IA) | 444 (IA) | 1690 |
| Palmitoylethanolamine | >1000 | >1000 | >1000 | 349 |

EC50 values in nM measured in GTPγ[³⁵S] binding assay
IA represents that the data describes represents inverse agonist activity.
All the ligands were purchased from Tocris.
Human CB1: Genebank accession number: U73304
Human CB1a: Genebank accession number: NM_{—}033181
Human CB2: Genebank accession number: NM_{—}001841

The difference in pharmacology illustrates the feasibility of identifying compounds that either have broad spectrum activity against more than one of the CB class of receptors, or which selectively targets one of these receptors (CB1, CB1a, CB1b or CB2), to trigger selective biological effects or to minimize side-effects associated with some of them.

1. An isolated nucleic acid molecule encoding a CB1b receptor, said nucleic acid molecule comprising a nucleotide sequence having at least 95% identity to a degenerate variant of SEQ ID NO: 1.

2. An isolated nucleic acid molecule encoding a CB1b receptor, said nucleic acid molecule comprising a nucleotide sequence having at least 95% identity to SEQ ID NO: 1.

3. An isolated nucleic acid molecule encoding a CB1b receptor comprising a nucleotide sequence of SEQ ID NO: 1.

4. The isolated nucleic acid molecule of claim 1, said nucleic acid molecule consisting of a nucleotide sequence of SEQ ID NO: 1.

5. An isolated nucleic acid molecule encoding a CB1a receptor comprising the amino acid sequence of SEQ ID NO: 2, or a sequence with 95% sequence identity thereto.

6. The isolated nucleic acid molecule of claim 5, wherein the nucleotide sequence encodes a polypeptide sequence consisting of the amino acid sequence of SEQ ID NO: 2

7. The nucleic acid molecule of claim 5, said nucleic acid molecule comprising a nucleotide sequence having at least 95% identity to a degenerate variant of SEQ ID NO: 1.

8. A vector comprising the nucleic acid molecule of claims 1 or 5.

9. A host cell comprising the vector of claim 8.

10. The cell of claim 9, wherein the cell expresses a polypeptide encoded by the nucleic acid molecule.

11. A purified polypeptide of the CB1b receptor comprising an amino acid sequence having at least 95% identity to the amino acid sequence of SEQ ID NO: 2.

12. The purified polypeptide of claim 11, wherein the amino acid sequence comprises the amino acid sequence of SEQ ID NO: 2.

13. A method for producing a CB1b receptor comprising:
a) culturing the host cell of claim 9 under conditions whereby said receptor is produced, and
b) recovering the receptor from the host cell culture or culture medium.

14. A method for detecting a polynucleotide which encodes a CB1b receptor in a biological sample comprising:
a) contacting a probe capable of selectively hybridising to CB1b nucleic acid with nucleic acid material of a biological sample, thereby forming a hybridization complex; and
b) detecting the hybridization complex, wherein the presence of the complex correlates with the presence of a polynucleotide encoding a CB1b receptor in the biological sample.

15. A method for detecting a polynucleotide which encodes a CB1b receptor in a biological sample comprising:
a) PCR amplification of nucleic acid from the biological sample using primers that hybridise either side of the 99 base deletion found in CB1b relative to CB1;
b) amplifying up the target region; and,
c) detecting the presence of a polynucleotide which encodes a CB1b receptor on the basis of the size of amplified product generated in step (b).

16. A method for identifying a compound which binds to the CB1b receptor, comprising
a) contacting the polypeptide of the CB1b receptor of claim 11 or 12 with a test compound, and
b) determining if the receptor binds to the test compound.

17. A purified antagonist, agonist, modulator or inverse agonist of the polypeptide of SEQ ID NO: 2.

18. A pharmaceutical composition comprising a substantially purified CB1b receptor inhibitory nucleic acid molecule, said molecule capable of selectively binding to the CB1b nucleic acid, in conjunction with a suitable pharmaceutical carrier.

19. The pharmaceutical composition of claim 18, wherein the inhibitory nucleic acid molecule is selected from: an antisense, a ribozyme, a triple helix and an RNAi molecule.

20. A pharmaceutical composition comprising an agonist, an inverse agonist, a modulator or an antagonist of the CB1b receptor of SEQ ID NO: 2.

21. A method for treating or preventing a CB associated disorder comprising administering to a subject in need of such treatment an effective amount of a pharmaceutical composition of claim 19 or 20.

22. A screening system wherein the modulatory ability of a test compound is determined by screening the compound against a panel of cannabinoid receptors, said panel comprising CB1b and at least one other cannabinoid receptor family member.

23. A screening system of claim 22, wherein the other cannabinoid receptor family member is selected from: CB1, CB1a and CB2.

24. A screening system of claim 23, wherein the test compound is screened against CB1b and at least CB1 and CB1a.

25. A screening system of claim 24, wherein the test compound is screened against CB1b and at least CB1, CB2 and CB1a.

26. A method for determining the selectivity of a test compound against a cannabinoid receptor family member, comprising determining the ability of the test compound to modulate each of a panel of cannabinoid receptors, said cannabinoid receptor panel comprising the CB1b receptor and at least one other cannabinoid receptor selected from: CB1, CB2 and CB1a.

27. The method of claim 26, wherein a profile of the modulatory ability of the test compound is compiled.

28. A method for identifying a compound which binds to the CB1b receptor, comprising:
a) contacting the cell of claim 10 with a test compound, and
b) determining if the receptor binds to the test compound.
